(19) United States
(12) Patent Application Publication
(10) Pub. No.: US 2017/0000179 A1
(43) Pub. Date: Jan. 5, 2017

(54) EDIBLE ADHESIVES AND INGESTIBLE COMPOSITIONS INCLUDING THE SAME

(71) Applicant: PROTEUS DIGITAL HEALTH, INC., Redwood City, CA (US)

(72) Inventors: Kevin Cheng, San Francisco, CA (US); Patricia Johnson, San Carlos, CA (US); Raymond Schmidt, San Mateo, CA (US); Anuj Patel, San Francisco, CA (US)

(73) Assignee: Proteus Digital Health, Inc., Redwood City, CA (US)

(21) Appl. No.: 15/113,032

(22) PCT Filed: Jan. 21, 2015
(86) PCT No.: PCT/US2015/012252

§ 371 Date: Jul. 20, 2016

Related U.S. Application Data
(60) Provisional application No. 61/930,399, filed on Jan. 22, 2014.

(51) Int. Cl. A23L 29/262; A61B 5/00; A61B 5/07

(52) U.S. Cl. CPC A23L 29/262; A61B 5/073; A61B 5/6861; A23V 2002/00

(57) ABSTRACT

Edible adhesives and ingestible compositions that include the same are provided. Aspects further include methods of making and using the compositions.

### CROSS-REFERENCE TO RELATED APPLICATIONS

This application claims priority to U.S. Provisional Application No. 61/930,399, filed Jan. 22, 2014, entitled EDIBLE ADHESIVES AND INGESTIBLE COMPOSITIONS INCLUDING THE SAME, the disclosure of which is incorporated in its entirety by this reference.

### INTRODUCTION

A variety of different ingestible compositions have been developed for nutritional, therapeutic and non-therapeutic uses. Examples of different types of ingestible compositions include orally ingestible tablets, capsules and liquids. A given orally ingestible formulation may include a variety of different components, such as active agents, carrier materials (including binders, bulking agents and other excipients), flavoring agents, coloring agents, etc. More recently, ingestible compositions which include a device component, such as an RFID tag or an ingestible event marker, have been developed.

As with many consumer products, ingestible compositions are not manufactured at the time of and location of use. Instead, they are generally manufactured at one or more fabrication facilities, stored for a period of time and then shipped to the end-user. Upon receipt, the end-user may further store them for a period of time before use.

During the multiple storage periods, and even manufacturing periods, such as mentioned above, the quality of the ingestible composition, e.g., in terms of effectiveness, may be degraded in some way. For example, exposure to humidity, elevated temperatures, microorganisms and oxidizing agents, as well other environmental hazards, can negatively impact the quality of the ingestible composition.

### SUMMARY

Edible Adhesives are provided. Aspects also include ingestible compositions that include the edible adhesives, a highly-swellable polymeric film and an ingestible component. Aspects further include methods of making and using the compositions.

### BRIEF DESCRIPTION OF THE FIGURES

FIG. 1 is a block diagram representation of one aspect of an ingestible event marker with dissimilar metals positioned on opposite sides of a support that includes a circuitry component.

FIG. 2 shows ionic transfer or the current path through a conducting fluid when the event indicator system of an ingestible event indicator is in contact with conducting liquid and in an active state. FIG. 2A shows an exploded view of the surface of a dissimilar material as illustrated in FIG. 2;

FIGS. 3A and 3B provide side and top views, respectively, of one aspect of an ingestible event indicator;

FIG. 4 is a block diagram representation of another aspect of the event indicator system with dissimilar metals positioned on the same end and separated by a non-conducting material.

FIG. 5 shows the event indicator system of FIG. 2 with a pH sensor unit.

FIG. 6 is a block diagram illustration of one aspect of the control device used in the system of FIGS. 1, 2 and 4.

FIG. 7 provides a view of an IEM that includes an edible adhesive composition, according to one aspect.

FIG. 8 provides a view of an edible adhesive composition on a release liner, according to one aspect.

FIGS. 9A and B provide depictions of two different lamination based methods of preparing an IEM as depicted in FIG. 7.

FIG. 10 is a functional block diagram of a demodulation circuit that performs coherent demodulation that may be present in a receiver, according to one aspect.

FIG. 11 illustrates a functional block diagram for a beacon module within a receiver, according to one aspect.

FIG. 12 is a block diagram of different functional modules that may be present in a receiver, according to one aspect.

FIG. 13 is a block diagram of a receiver, according to one aspect.

FIG. 14 provides a block diagram of a high frequency signal chain in a receiver, according to one aspect.

FIG. 15 provides a diagram of how a system that includes a signal receiver and an ingestible event marker may be employed, according to one aspect.

### DETAILED DESCRIPTION

Edible Adhesives are provided. Aspects also include ingestible compositions that include the edible adhesives, a highly-swellable polymeric film and an ingestible component. Aspects further include methods of making and using the compositions.

### Compositions

As summarized above, aspects of the invention include edible adhesives as well as compositions which include these edible adhesives in addition to other components, e.g., ingestible components such as ingestible devices, minimally dimensioned components, highly-swellable polymeric films; etc.

### Edible Adhesives

Aspects of the invention include edible adhesive compositions. As the compositions are edible, they are suitable for ingestion by a living animal, e.g., a vertebrate, including a mammal, such as a human. Accordingly, the adhesive compositions are non-toxic to the living animal, at least in the amounts at which the adhesive is present in the ingestible composition of which it is a part. By non-toxic is meant not capable of causing harm, at least for a normal subject not having an allergy to an ingredient of the adhesive composition.

As the compositions are adhesive, when positioned between two materials, e.g., a film and an ingestible components, such as described below, the compositions cause the two objects to adhere to each other. Accordingly, when an adhesive composition is positioned between two objects, the two objects stay attached to each other, i.e., stick fast to, cleave to, or cling to, each other. The strength of the adhesive compositions may vary, and in some instances is sufficiently strong such that two objects adhered to each other with the adhesive stay adhered to each other under pharmaceutical storage conditions, e.g., in a sterile packaging at standard temperature/pressure. While the adhesive strength of the compositions may vary, in some instances the adhesives have a strength as measured by the T-Peel test that ranges from 1 mN to 100 mN . An example of a T-Peel test that may be employed to determine adhesive strength is ASTM D1876.

Edible adhesive compositions of interest include a cellulose ether, a tackifier and a plasticizer. Of interest as cellulose ethers are nonionic cellulose ethers. Examples of such cellulose ethers include, but are not limited to: methylcellulose, ethylcellulose, hydroxymethylcellulose, hydroxyethylcellulose, carboxymethylcellulose, carboxyethylcellulose and hydroxypropylcellulose, hydroxypropyl methyl cellulose; additional celluloses, such as carboxymethylcellulose sodium, carboxymethylcellulose calcium, carboxymethylstarch; etc. In certain embodiments, the cellulose ether is a hydroxypropyl cellulose. Hydroxypropyl cellulose is available from a variety of commercial sources, including but limited to, Nisso America Inc. In certain embodiments, the average molecular weight of the hdyroxypropyl cellulose is 10,000 daltons or higher, such as 25,000 daltons or higher, where the average molecular weight may be as high as 40,000 daltons or higher, e.g., 50,000 daltons or higher, 100,000 daltons or higher, and including 250,000 daltons or higher, 500,000 daltons or higher, 750,000 daltons or higher, such as 1,000,000 daltons or higher, where in some instances the molecular weight ranges from 10,000 to 1,000,000 daltons, such as 25,000 to 500,000 daltons, including 30,000 to 250,000 daltons. While the degree of substitution may also vary, in some instances the cellulose has a degree of substitution ranging from 50 to 80%. The proportion of the cellulose ether in the adhesive compositions may vary, ranging in some instances from 50 to 95%, (w/w), such as from 60 to 90% (w/w), including from 70 to 85% (w/w).

Adhesives of interest further include a tackifying agent, i.e., a tackifier. Tackifying agents of interest include, but are not limited to: natural, semi-synthetic and synthetic water-soluble tackifiers. Examples of the natural tackifiers include xanthan gum, xanthane flow, pectin from vegetables, gum arabic and guar gum. Examples of the semi-synthetic tackifiers include methylated products of cellulose or starch derivatives, their carboxyalkylated products and hydroxyalkylated products and examples of the synthetic tackifiers include polyacrylates, polymaleates, polyvinylpyrrolidone, and the like. In some instances, the tackyfying agent is a polyvinylpyrrolidone (i.e., povidone), which is synthetic polymer consisting mainly of linear 1-vinyl-2-pyrrolidone groups. Povidones (which may be cross-linked) are available from a variety of different commercial sources, including but not limited to: Sigma, BASF and the like. In some instances, the povidone has a molecular weight ranging from 35 kilodaltons to 45 kilodaltons. The proportion of the tackifying agent in the adhesive may vary, ranging in some instances from 1 to 25%, (w/w), such as from 3 to 20% (w/w), including from 5 to 15% (w/w), e.g., from 5 to 10% (w/w).

Also present in the adhesive compositions is a plasticizer. Plasticizers of interest include glycerin, polyethylene glycol, triethylcitrate, and dibutyl sebacate. In some instances, the plasticizer is glycerin. The proportion of the plasticizer in the adhesive may vary, ranging in some instances from 5 to 30%, (w/w), such as from 7.5 to 25% (w/w), including from 10 to 20% (w/w).

While the edible adhesive compositions described herein may find use in a variety of applications, there are suited for use in adhering highly-swellable polymeric films to ingestible components. As such, each of these two classes of objects that can be adhered to each other with adhesives described herein are now reviewed in greater detail.

### Highly-Swellable Polymeric Films

In some instances, the edible adhesive is present in a composition that also includes a highly-swellable polymeric film. As used herein, the term “film” means a thin sheet or layer. While the dimensions of the film may vary, in some instances the film has a thickness of 10 microns or greater, such as 50 microns or greater, including 100 microns or greater, and ranges in thickness in some instances from 10 to 1000, such as 20 to 200 and including 30 to 60 microns. The top and bottom surfaces of the film may have a variety of different configurations, including but not limited to rectangular, trapezoidal, triangular, etc.; curvilinear, such as circular, ovoid or other curvilinear shape, etc. Where the film has surface which may be defined by length and width, these dimensions may vary, where in some instances the length ranges from 1 to 20, such as 2 to 10 and including 3 to 6 mm and the width ranges from 1 to 20, such as 2 to 10 and including 3 to 6 mm.

Films of interest are highly-swellable. By highly-swellable is meant that that the films are able to swell substantially upon contact with a liquid aqueous medium, such that they grow substantially in bulk (i.e., magnitude in three dimensions, e.g., which may be assessed in terms of a change in volume, etc.) by the absorption of water upon contact with an aqueous medium. Where the swelling results in a change in volume of the film, the volume may increase by a factor of 10 or greater, such as a factor of 15 or greater, including a factor of 20 or greater, e.g., a factor of 25 or greater, a factor of 30 or greater, a factor of 40 or greater, including a factor of 50 or greater, such as a factor of 100 or greater, for example a factor of 500 or greater, including a factor of 1000 or greater, as compared to the initial volume prior to contact with the liquid aqueous medium.

Upon swelling, the mass of film may increase as well, where in some instances the mass increases by a factor of 10 or greater, such as a factor of 15 or greater, including a factor of 20 or greater, e.g., a factor of 25 or greater, a factor of 30 or greater, a factor of 40 or greater, including a factor of 50 or greater, such as a factor of 100 or greater, for example a factor of 500 or greater, including a factor of 1000 or greater, as compared to the initial mass prior to contact with the aqueous medium.

Highly-swellable polymeric films described herein rapidly swell upon contact with a liquid aqueous medium. By “rapidly swell” is meant that upon contact with a liquid aqueous medium, the films achieve substantially maximum swelling in a short period of time. As such, following contact with an aqueous medium, the films achieve 90% or more, such as 95% percent or more maximal swelling in a period of time of 10 minutes or less, such as 5 minutes or less, including 1 minute or less. In some instances, the films swell in volume by a factor of 10 or greater, such as a factor of 15 or greater, including a factor of 20 or greater, e.g., a factor of 25 or greater, a factor of 30 or greater, a factor of 40 or greater, including a factor of 50 or greater, such as a factor of 100 or greater, for example a factor of 500 or greater, including a factor of 1000 or greater, as compared to the initial volume prior to contact with the aqueous medium, in 10 minutes or less, such as 5 minutes or less, including 1 minute or less.

As mentioned above, films of interest are configured to absorb water upon contact with an aqueous medium. While the amount of water that is absorbed by a given film may vary, in some instances the films absorb 10 or more grams of water per gram dry weight of film, such as 25 or more grams of water per gram of dry weight of film, including 50 or more grams of water per gram of dry weight film, upon contact with an aqueous medium.

Highly-swellable polymeric films of interest are those that swell substantially upon contact with a liquid aqueous medium, but do not swell substantially, if at all, upon contact with a gaseous medium that includes water vapor. As such, upon contact with a gaseous medium that includes water vapor (e.g., where the partial pressure of water ranges from 1.0 to 49.8, such as 2.7 to 21.4 mmHg), the films swell little, if at all. As such, any swelling that occurs upon contact with such a gaseous medium as determined by a change in volume is a factor of five or less, such as a factor of 2 or less as compared to the film prior to contact with the gaseous medium.

Highly swellable polymeric films of interest exhibit rapidly swelling behavior without disintegrating upon contact with a liquid aqueous medium. As such, the films swell upon contact with an aqueous medium but do not break up or separate into parts, such that they do not lose intactness or solidness, at least not for a prolonged period of time, such as 1 hour or longer, e.g., 2 hours or longer, including 3 hours or longer, such as 5 hours or longer, including 6, 12, 18, 24 , 36, 48, 60, or 72 hours or longer, wherein in some instances the films may be fabricated not to disintegrate, e.g., dissolve, for a period of time ranging from 1 to 100, such as 5 to 75 hours following contact with an aqueous medium. As such, they do not dissolve upon contact with a liquid aqueous medium. Accordingly, a film that contacts a liquid aqueous medium will remain as a single entity following swelling, and will not go into solution. Therefore, following contact with a liquid aqueous medium, the film can still be manipulated, i.e., handled.

In some instances, the highly-swellable polymeric films are ingestible. As such films are ingestible, they are configured to be ingested or swallowed, i.e., taken into the stomach by drawing through the throat and esophagus with a voluntary muscular action. As such, the films themselves, as well as the components thereof, e.g., polymeric components, binders, plasticizers, porogens (such as described in greater detail below), do not exhibit an unacceptable level of toxicity when employed as intended. In other words, when the films are employed for their intended use, the toxicity level of the films, if present all, is acceptable.

Prior to contact with an aqueous medium, the freestanding films are mechanically stable. Films are flexible, able to easily wrap around rollers and rods and relatively strong under tension, but show little elongation under strain.

Films of interest may include an ionic polymer and may therefore be referred to as polymeric films. The phrase “ionic polymer” refers to a polymer comprising monomeric units having an acidic functional group, such as a carboxyl, sulfate, sulfonate, phosphate or phosphonate group, or a basic functional group, such as an amino, substituted amino or guanidyl group. When in aqueous solution at a suitable pH range, e.g., 7 to 14 pH, an ionic polymer comprising acidic functional groups will be a polyanion, and such a polymer is referred to herein as an “anionic polymer”. Likewise, in aqueous solution at a suitable pH range, e.g., 1 to 7 pH, an ionic polymer comprising basic functional groups will be a polycation. Such a polymer is referred to herein as a “cationic polymer”. As used herein, the terms ionic polymer, anionic polymer and cationic polymer refer to hydrophilic polymers in which the acidic or basic functional groups are not charged, as well as polymers in which some or all of the acidic or basic functional groups are charged, in combination with a suitable counterion. Suitable anionic polymers include alginates, e.g., alginic acid and salts thereof, polyacrylic acid, dextran sulfate, carboxymethylcellulose, hyaluronic acid, polyglucuronic acid, polymanuronic acid, polygalacturonic acid, polyarabinic acid; chrondroitin sulfate and dextran phosphate. Suitable cationic polymers include chitosan, polyethylenimine, poly-L-lysine, and dimethylaminodextran. Of interest in some instances are polysaccharide anionic polymers. Polysaccharide anionic polymers of interest include alginates, e.g., alginic acid and salts thereof. Alginic acid (i.e., alginate) is a linear copolymer with homopolymeric blocks of (1-4)-linked β-D-mannuronate (M) residues and α-L-guluronate (G) residues. The residues are covalently linked together in different sequences or blocks. The residues can appear in homopolymeric blocks of consecutive G-residues (G-blocks), consecutive M-residues (M-blocks) or alternating M and G-residues (MG-blocks). Also of interest are salts of alginic acid, e.g., sodium alginate, calcium alginate, potassium alginate, etc. The molecular weight of the alginate (e.g., alginic acid or alginate salt thereof) may vary, ranging in some instances from 10,000 to 600,000 Daltons, such as 50,000 to 100,000 Daltons. Alginates of interest will include a percentage of acidic groups sufficient to impart the above described swellability characteristic to the film. As such, where an alginate is employed that does not initially include the desired acidic groups, it may be modified as necessary to provide for the desired acidic groups. For example, where sodium alginate is employed, some of the sodium groups of the sodium alginate may be converted to acidic groups, e.g., by contacting the film with a suitable acid (such as NCI). To impart the desired mechanical properties to the film, the alginates may be cross-linked. For example, where sodium alginate is employed, the alginate may be cross-linked with a divalent cation salt, e.g., calcium chloride, magnesium chloride, etc.

Where desired, the film may include an additional component that provides for acidic, e.g., carboxyl, functional groups. For example, films of interest may include one or more additional polymers that provide for acidic functional groups, e.g., one or more additional anionic polymers that are present in addition to an alginate. Examples of additional anionic polymers of interest that may be present include, but are not limited to both natural and synthetic polymers.

In some instances, the film is a blend of both an alginate and a polyacrylic acid. Polyacrylic acids of interest include both homopolymeric polyacrylic acid as well as copolymers, including both random and block copolymers, of acrylic acid residues and one or more non-acrylic acid residues, e.g., acrylate residues, etc. Where desired, the polyacrylic acid may be cross-linked. When the film includes a blend of alginate to polyacrylic acid polymers, the dry weight ratio of the two types of polymers in the film may vary, and in some instances ranges from 25 to 95%, such as 50 to 80% and including 70 to 80% alginate.

In addition to the polymeric components, the films may further include one or more additional types of components. For example, films may include one or more agents that enhance conductivity of the film upon contact with an aqueous medium. Examples of such components include pore forming agents (i.e., porogens). The term “porogen” as used herein, refers to a chemical compound that is included in the film and, upon contact with an aqueous medium, is removed from the film, e.g., via diffusion, dissolution, and/or degradation, to leave a pore in the resultant film. The diameter of the pores produced by the porogen may vary, ranging in some instances from 1 to 1000 μm, such as 1 to 500 μm and including 1 to 250 μm. Porogens of interest include both inorganic and organic porogens. Inorganic porogens of interest include, but are not limited to: inorganic salts, e.g., NaCl, MgCl₂, CaCl₂, NH₄Cl, NH₄PO₄, NH₄CO₃, soluble biocompatible salts; sugars (e.g., sugar alcohols), polysaccharides (e.g., dextran (poly(dextrose)), water soluble small molecules, natural or synthetic polymers, oligomers, or monomers that are water soluble or degrade quickly under physiological conditions, including but not limited to: polyethylene glycol, polyvinyl alcohol, poly(vinylpyrollidone), pullulan, poly(glycolide), poly(lactide), poly(lactide-co-glycolide), other polyesters, and starches. When present, the total amount of porogen component made up of one or more porogens may vary. In some instances, the amount of porogen component ranges from 1 to 40, including from 5 to 10 dry weight percent of the film.

Where desired, films may include one or more binding agents or binders. Binders of interest include, but are not limited to: celluloses, e.g., methyl cellulose, hydroxymethyl cellulose, hydroxyethyl cellulose, hydroxypropyl cellulose, hydroxypropyl methylcellulose, etc.; polyvinyl pyrrolidone; polyethylene oxides; gums, acrylate polymers; methacrylate polymers; copovidone; etc.

When present, the total amount of binder component made up of one or more binding agents may vary. While the total amount of one or more binding agents in the film may vary, in some instances the amount ranges from 1 to 50, such as 5 to 10 dry weight percent of the film.

Where desired, films may include one or more plasticizing agents.

Plasticizing agents of interest include, but are not limited to: fatty acids, e.g., oleic acid, palmitic acid, etc.; dioctylphtalate; phospholipid; phosphatidic acid; polyethylene glycol; glycerine; butylhydroxytoluene; salts; saccharides, e.g., sucrose; etc. When present, the total amount of plasticizer component made up of one or more plasticizing agents may vary. In some instances, the amount of plasticizer component ranges from 0.01 to 10, including from 2 to 5 dry weight percent of the film.

Highly-swellable polymeric films of interest are further described in United States Provisional Application Ser. No. 61/758,030 filed on Jan. 29, 2013; the disclosure of which is herein incorporated by reference.

### Ingestible Compositions

Aspects of the invention include ingestible compositions. In these instances, ingestible compositions of interest include both an ingestible component and a highly-swellable film component which is associated therewith by an edible adhesive, e.g., as described above. As the compositions are ingestible, they are configured to be ingested or swallowed, i.e., taken into the stomach by drawing through the throat and esophagus with a voluntary muscular action. Accordingly, the compositions are dimensioned so as to be capable of being ingested. In some instances, the compositions have a longest dimension of 30 mm or less, such as 20 mm or less, e.g., 10 mm or less. The volume of the ingestible composition may also vary so long as the composition is suitable for ingestion, where the volume in some instances may be 25 mm³ or less, such as 15 mm³ or less, including 10 mm³ or less.

In the ingestible compositions, the ingestible component is a portion or part of the ingestible composition that is configured for ingestion. The ingestible component may vary widely and may include one or more subcomponents, e.g., a pharmaceutically acceptable solid carrier (which may or may not include an active agent), a device (which may or may not include electronic circuitry), etc. In the ingestible composition, the highly-swellable polymeric film may be associated with an ingestible composition in a number of different ways, e.g., depending on the nature of the ingestible composition, depending on the purpose of the film, etc.

In some instances, the ingestible component includes a pharmaceutically acceptable solid carrier. Pharmaceutically acceptable solid carrier configurations include tablet and capsule configurations. While the pharmaceutically acceptable solid carrier may have a solid configuration, the solid configuration may include a liquid component, such as is found in a liquid capsule, which includes a liquid component present in a solid capsule. In some instances, the pharmaceutically acceptable solid carrier is configured to impart a controlled release profile to an active agent that is associated with the pharmaceutically acceptable solid carrier. Examples of pharmaceutically acceptable solid carriers of interest can be found in Remington's Pharmaceutical Sciences, Mace Publishing Company, Philadelphia, Pa., 17th ed. (1985). In such instances, among other non-mutually exclusive functions, e.g., as described below, the highly-swellable polymeric films may serve to separate the ingestible component from the carrier. For instance, upon contact with an aqueous medium, the resultant swelling of the film may result in the component to which the film is associated being pushed away and separated from the carrier, e.g., tablet or capsule. Such functionality of the film may be desirable, e.g., where association of the carrier material impedes functionality of the ingestible component (such as by blocking access of electrode material to an aqueous environment).

Where desired, the pharmaceutically acceptable solid carrier may include an active agent. Active agents of interest include pharmaceutically active agents as well as non-pharmaceutical active agents, such as diagnostic agents. The phrase “pharmaceutically active agent” (also referred to herein as drugs) refers to a compound or mixture of compounds which produces a physiological result, e.g., a beneficial or useful result, upon contact with a living organism, e.g., a mammal, such as a human. Pharmaceutically active agents are distinguishable from such components as excipients, carriers, diluents, lubricants, binders and other formulating aids, and encapsulating or otherwise protective components. The pharmaceutically active agent may be any molecule, as well as binding portion or fragment thereof, that is capable of modulating a biological process in a living subject. In certain aspects, the pharmaceutically active agent may be a substance used in the diagnosis, treatment, or prevention of a disease or as a component of a medication. The pharmaceutically active agent is capable of interacting with a target in a living subject. The target may be a number of different types of naturally occurring structures, where targets of interest include both intracellular and extracellular targets. Such targets may be proteins, phospholipids, nucleic acids and the like, where proteins are of particular interest. Specific proteinaceous targets of interest include, without limitation, enzymes, e.g., kinases, phosphatases, reductases, cyclooxygenases, proteases and the like, targets comprising domains involved in protein-protein interactions, such as the SH2, SH3, PTB and PDZ domains, structural proteins, e.g., actin, tubulin, etc., membrane receptors, immunoglobulins, e.g., IgE, cell adhesion receptors, such as integrins, etc., ion channels, transmembrane pumps, transcription factors, signaling proteins, and the like. Broad categories of active agents of interest include, but are not limited to: cardiovascular agents; pain-relief agents, e.g., analgesics, anesthetics, anti-inflammatory agents, etc.; nerve-acting agents; chemotherapeutic (e.g., anti-neoplastic) agents; neurological agents, e.g., anti-convulsants, etc. The amount of active agent that is present in the solid carrier may vary. In some instances, the amount of active agent that is present may range from 0.01 to 100% by weight.

Further examples of pharmaceutically acceptable solid carriers and active agents which may or may not be included therein are described in PCT Application Serial No. PCT/US2006/016370 published as WO/2006/116718, PCT Application Serial No. PCT/US2007/082563 published as WO/2008/052136, PCT Application Serial No. PCT/US2007/024225 published as WO/2008/063626, PCT Application Serial No. PCT/US2007/022257 published as WO/2008/066617, PCT Application Serial No. PCT/US2008/052845 published as WO/2008/095183, PCT Application Serial No. PCT/US2008/053999 published as WO/2008/101107, PCT Application Serial No. PCT/US2008/056296 published as WO/2008/112577, PCT Application Serial No. PCT/US2008/056299 published as WO/2008/112578, PCT Application Serial No. PCT/US2008/077753 published as WO2009/042812; PCT Application Serial No. PCT/US2008/085048 published as WO2009/070773; PCT Application Serial No. PCT/US2009/36231 published as WO2009/111664; PCT Application Serial No. PCT/US2009/049618 published as W02010/005877; PCT Application Serial No. PCT/US2009/053721 published as WO2010/019778; PCT Application Serial No. PCT/US2009/060713 published as WO2010/045385; PCT Application Serial No. PCT/US2009/064472 published as WO2010/057049; PCT Application Serial No. PCT/US2009/067584 published as WO2010/068818; PCT Application Serial No. PCT/US2009/068128 published as WO2010/075115; PCT Application Serial No. PCT/US2010/020142 published as WO2010/080765; PCT Application Serial No. PCT/US2010/020140 published as WO2010/080764; PCT Application Serial No. PCT/US2010/020269 published as WO2010/080843; PCT Application Serial No. PCT/US2010/028518 published as WO2010/111403; PCT Application Serial No. PCT/US2010/032590 published as WO2010/129288; PCT Application Serial No. PCT/US2010/034186 published as WO2010/132331; PCT Application Serial No. PCT/US2010/055522 published as WO2011/057024; the disclosures of which are herein incorporated by reference.

With such ingestible compositions, the highly-swellable polymeric film may be associated with the ingestible component in a number of different ways. For example, the highly-swellable polymeric film may form a coating or layer that substantially if not completely encloses the ingestible component. Such a configuration may be employed where it is desired to impart shelf-life stability to the ingestible composition. In such instances, the film imparts shelf-life stability to the composition, in that the film enhances the storage stability of the composition by a quantifiable measure as compared to a control or reference composition (i.e., a composition that lacks the shelf-life stability component). Highly-swellable polymeric films of interest may enhance the shelf-life stability of the composition as compared to a suitable control by a magnitude of 2-fold or greater, such as 5-fold or greater including 10-fold or greater, e.g., 25-fold or greater. The presence of the film allows the composition to be stable for extended periods of time during or following manufacture, where the ingestible composition may be stable for one year or longer, such as two years or longer, including five years or longer, following manufacture when the composition is maintained under conditions in which the temperature ranges from 10 to 40° C., the pressure ranges from 0.5 to 2.0 ATM and the relative humidity ranges from 10 to 100%. By “stable” is meant that the functionality of the composition does not degrade to a point that the composition is no longer suitable for use in its intended purpose. For example, if the composition includes an active pharmaceutical agent, the amount of active agent following the storage time period may be 85% or more, such as 90% or more, including 95% or more of the original amount present in the composition following manufacture, e.g., as determined using an HPLC protocol or other suitable analytical technique which can distinguish the amount of active agent from any degradation byproducts, such as oxidation byproducts.

In addition to or instead of a pharmaceutically acceptable solid carrier, ingestible compositions may include a device. The term “device” is used broadly to refer to a mechanical and/or electrical component configured for a particular purpose, where the device may or may not include a circuitry component.

Of interest as devices are ingestible devices, e.g., RFID-enabled devices; ingestible event indicators (also known as ingestible event markers or IEMS), etc. An ingestible event indicator is a device that is dimensioned to be ingestible and includes an identifier circuitry component and, optionally, a current path extender, e.g., a membrane, sometimes referred to herein as a “skirt”. Various aspects of an event indicator may include a control device for altering conductance; and a partial power source. The partial power source may include a first material electrically coupled to the control device; and a second material electrically coupled to the control device and electrically isolated from the first material, where the first and second materials are dissimilar.

Upon ingestion, the event indicator contacts a conducting fluid, e.g., stomach fluid. When the event indicator is in contact with the conducting liquid, a current path is formed through the conducting liquid between the first and second materials. The voltage potential created between the materials provides the power for operating the event indicator as well as produces the current flow through the conducting fluid and the system. In one aspect, the event indicator operates in direct current mode. In an alternative aspect, the event indicator controls the direction of the current so that the direction of current is reversed in a cyclic manner, similar to alternating current. The current path through the system is controlled by the control device. Completion of the current path allows for the current to flow and in turn a receiver, not shown, can detect the presence of the current and recognize that the system has been activated and the desired event is occurring or has occurred.

In one aspect, the two materials are similar in function to the two electrodes needed for a direct current power source, such as a battery. The conducting liquid acts as the electrolyte needed to complete the power source. The completed power source is defined by the electrochemical reaction between the dissimilar materials of the event indicator and the completion of the power source is enabled by the fluids of the body. The completed power source may be viewed as a power source that exploits electrochemical conduction in an ionic or a conducting solution such as gastric fluid, blood, or other bodily fluids and some tissues.

In certain aspects, the complete power source or supply is one that is made up of active electrode materials, electrolytes, and inactive materials, such as current collectors and packaging. The active materials are any pair of materials with different electrochemical potentials. Suitable materials are not restricted to metals, and in certain aspects the paired materials are chosen from metals and non-metals, e.g., a pair made up of a metal (such as Mg) and a salt (such as Cul). With respect to the active electrode materials, any pairing of substances—metals, salts, or intercalation compounds—with suitably different electrochemical potentials (voltage) and low interfacial resistance are suitable. Where desired, the voltage provided by the two dissimilar electrochemical materials upon contact of the materials of the power source with the target physiological site is 0.001 V or higher, including 0.01 V or higher, such as 0.1 V or higher, e.g., 0.3 V or higher, including 0.5 volts or higher, and including 1.0 volts or higher, where in certain aspects, the voltage ranges from about 0.001 to about 10 volts, such as from about 0.01 to about 10 V.

Anode materials of interest include, but are not limited to: magnesium, zinc, sodium, lithium, iron and alloys thereof, e.g., Al and Zn alloys of Mg, which may or may not be intercalated with a variety of materials such, as graphite with Li, K, Ca, Na, Mg, and the like. Cathode materials of interest include, but are not limited to, copper salts, such as copper salts of iodide, chloride, bromide, sulfate, formate, Fe³⁺ salts, e.g., orthophosphate, pyrophosphate, silver salts, etc. One or both of the metals may be doped with a non-metal, for example to enhance the voltage output of the battery. Non-metals that may be used as doping agents in certain aspects include, but are not limited to: sulfur, iodine and the like. In certain aspects, the electrode materials are cuprous iodine (Cul) or cuprous chloride (CuCl) as the anode and magnesium (Mg) metal or magnesium alloy as the cathode. Aspects of the present invention use electrode materials that are not harmful to the human body.

In such ingestible compositions, the film may be associated with the event indicator in a number of different ways, where the different ways are not mutually exclusive such that films may be associated with an event indicator in more than one way. For example, a highly-swellable polymer film may cover a portion of at least one of the first or second dissimilar materials. As such, the film may cover a portion or all of the cathode material. Alternatively or in addition, the film may cover a portion or all of the anode material. In such instances, the film may cover 10% or more, 20% or more, 25% or more, 50% or more, including 75% or more, e.g., 90% or more, etc., of the cathode and/or anode materials, including all of the cathode or anode materials.

With respect to current signatures produced by such event indicators, the current signatures may distinguish one class of event indicator from other types or may be universally unique, such as where the current signature is analogous to a human fingerprint which is distinct from any other fingerprint of any other individual and therefore uniquely identifies an individual on a universal level. In various aspects, the control circuit may generate a variety of different types of communications, including but not limited to: RF signals, magnetic signals, conductive (near-field) signals, acoustic signals, etc.

In various aspects, the event indicator may further include a current path extender, such as a membrane, which produces a virtual dipole length between the pair of dissimilar materials (functioning as transmission elements) that is longer than the actual dipole length. In addition to controlling the magnitude of the current path between the materials, such a membrane (sometimes referred to herein as “amplifier” or “skirt”) is used to increase the “length” of the current path and, hence, act to boost the conductance path, as disclosed in the PCT application no. PCT/US2008/077753 published as WO2009/042812 and in U.S. Pat. No. 7,978,064, the entire contents of which are incorporated herein by reference. Throughout the disclosure herein, the terms “membrane”, “skirt” and “amplifier” are used interchangeably with the term “current path extender” without impacting the scope or the present aspects and the claims herein.

Where desired, an ingestible event indicator may be stably associated in some manner to another ingestible component, e.g., pharmaceutically acceptable carrier component (e.g., as described above). By “stably associated” is meant that the event indicator and second ingestible component, e.g., a pharmaceutically acceptable carrier component, do not separate from each other, at least until administered to the subject in need thereof, e.g., by ingestion. As the event indicators are dimensioned to be ingestible, they are sized so that they can be placed in a mammalian, e.g., human or animal, mouth and swallowed. In some instances, event indicators have a longest dimension that is 30 mm or less, such as 20 mm or less, including 5 mm or less.

Various aspects of ingestible event indicators of interest (including protocols for the fabrication thereof) are described in PCT Application Serial No. PCT/US2006/016370 published as WO/2006/116718, PCT Application Serial No. PCT/US2007/082563 published as WO/2008/052136, PCT Application Serial No. PCT/US2007/024225 published as WO/2008/063626, PCT Application Serial No. PCT/US2007/022257 published as WO/2008/066617, PCT Application Serial No.

PCT/US2008/052845 published as WO/2008/095183, PCT Application Serial No. PCT/US2008/053999 published as WO/2008/101107, PCT Application Serial No. PCT/US2008/056296 published as WO/2008/112577, PCT Application Serial No. PCT/US2008/056299 published as WO/2008/112578, PCT Application Serial No. PCT/US2008/077753 published as WO2009/042812; PCT Application Serial No. PCT/US2008/085048 published as WO2009/070773; PCT Application Serial No. PCT/US2009/36231 published as WO2009/111664; PCT Application Serial No.

PCT/US2009/049618 published as WO2010/005877; PCT Application Serial No. PCT/US2009/053721 published as WO2010/019778; PCT Application Serial No. PCT/US2009/060713 published as WO2010/045385; PCT Application Serial No. PCT/US2009/064472 published as WO2010/057049; PCT Application Serial No. PCT/US2009/067584 published as WO2010/068818; PCT Application Serial No.

PCT/US2009/068128 published as WO2010/075115; PCT Application Serial No. PCT/US2010/020142 published as WO2010/080765; PCT Application Serial No. PCT/US2010/020140 published as WO2010/080764; PCT Application Serial No. PCT/US2010/020269 published as WO2010/080843; PCT Application Serial No. PCT/US2010/028518 published as WO2010/111403; PCT Application Serial No.

PCT/US2010/032590 published as WO2010/129288; PCT Application Serial No. PCT/US2010/034186 published as WO2010/132331; PCT Application Serial No. PCT/US2010/055522 published as WO2011/057024; the disclosures of which are herein incorporated by reference.

In certain aspects, the ingestible event indicators are disrupted upon administration to a subject. As such, in certain aspects, the compositions are physically broken, e.g., dissolved, degraded, eroded, etc., following delivery to a body, e.g., via ingestion, injection, etc. The compositions of these aspects are distinguished from devices that are configured to be ingested and survive transit through the gastrointestinal tract substantially, if not completely, intact.

Where desired, an active agent (e.g., as described above) may be present in one or more of the event indicator components, e.g., in the electrochemical materials, the support, the membrane, etc. Examples of such configurations are described in PCT Application Serial No. PCT/US2010/032590 published as WO2010/129288; the disclosures of which are herein incorporated by reference.

In some instances the membrane may be made up partially or completely of a highly-swellable polymeric film. For example, the entire membrane may be fabricated from a highly-swellable polymeric film. Alternatively, a portion of the membrane, such as the outer-periphery of the membrane, may be made up of the highly-swellable polymeric film, with the remainder of the membrane being made up of one or more other suitable materials.

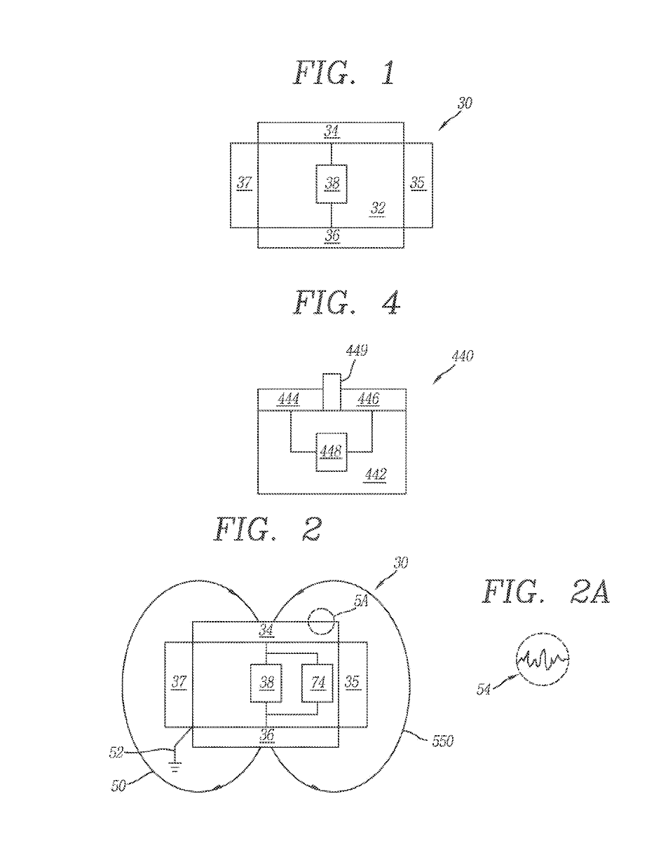

With reference to FIG. 1, there is shown one aspect of an ingestible device event indicator system with dissimilar metals positioned on opposite sides of system 30. The system 30 can be used in association with any pharmaceutical product, as mentioned above, and in one aspect, to determine when a patient takes the pharmaceutical product. The scope of the present invention is not limited by the environment and the product that is used with the system 30. For example, the system 30 may be placed within a capsule and the capsule is placed within the conducting liquid. The capsule would then dissolve over a period of time and release the system 30 into the conducting liquid. Thus, in one aspect, the capsule would contain the system 30 and no product. Such a capsule may then be used in any environment where a conducting liquid is present and with any product. For example, the capsule may be dropped into a container filled with jet fuel, salt water, tomato sauce, motor oil, or any similar product. Additionally, the capsule containing the system 30 may be ingested at the same time that any pharmaceutical product is ingested in order to record the occurrence of the event, such as when the product was taken.

In the specific example of the system 30 combined with the pharmaceutical product, as the product or pill is ingested, the system 30 is activated. The system 30 controls conductance to produce a unique current signature that is detected, thereby signifying that the pharmaceutical product has been taken. In other aspects, the current signature may contain information on the ingredients of the ingested pharmaceutical product which may include their chemical composition, date of manufacture, batch number, etc., among other desired information related to the pharmaceutical product which may be a placebo as well. The system 30 includes a framework 32. The framework 32 is a chassis for the system 30 and multiple components are attached to, deposited upon, or secured to the framework 32. In this aspect of the system 30, a digestible material 34 is physically associated with the framework 32. The material 34 may be chemically deposited on, evaporated onto, secured to, or built-up on the framework all of which may be referred to herein as “deposit” with respect to the framework 32. The material 34 is deposited on one side of the framework 32. The materials of interest that can be used as material 34 include, but are not limited to those described above. The material 34 is deposited by using any convenience protocol, including but not limited to physical vapor deposition, electro-deposition, or plasma deposition, liquid deposition, among other protocols. The material 34 may be from about 0.05 to about 500 μm thick, such as from about 5 to about 100 μm thick. The shape may be controlled by shadow mask deposition, or photolithography and etching. Additionally, even though only one region is shown for depositing the material, each system 30 may contain two or more electrically unique regions where the material 34 may be deposited, as desired. It should be noted that while the electrode materials described in connection with FIG. 1 are discussed above in terms of a particular fabrication protocol, they may be fabricated using other protocols, such as the laminate or printing protocols described in greater detail below in connection with the discussion of large surface area electrodes.

At a different side, which may be the opposite side as shown in FIG. 2, another digestible material 36, electrically isolated from the material 34, is deposited, such that materials 34 and 36 are dissimilar. Although not shown, the different side selected may be the side next to the side selected for the material 34. The scope of the present invention is not limited by the side selected and the term “different side” can mean any of the multiple sides that are different from the first selected side. Furthermore, even though the shape of the system is shown as a square, the shape may be any geometrically suitable shape. Materials 34 and 36 are selected such that they produce a voltage potential difference when the system 30 is in contact with conducting liquid, such as body fluids. The materials of interest for material 36 include, but are not limited to: Mg, Zn, or other electronegative metals, e.g., as described above. As indicated above with respect to the material 34, the material 36 may be chemically deposited on, evaporated onto, secured to, or built-up on the framework. Also, an adhesion layer may be employed, as convenient, to help the material 36 (as well as material 34 when needed) to adhere to the framework 32. Adhesion layers of interest for the material 36 are Ti, TiW, Cr or similar material. Anode material and the adhesion layer may be deposited by physical vapor deposition, electro-deposition or plasma deposition, where desired. The material 36 may be from about 0.05 to about 500 μm thick, such as from about 5 to about 100 μm thick. However, the scope of the present invention is not limited by the thickness of any of the materials nor by the type of process used to deposit or secure the materials to the framework 32.

Thus, when the system 30 is in contact with the conducting fluid, e.g., a liquid, a current path, an example is shown in FIG. 2, is formed through the conducting liquid between material 34 and 36. A control device 38 is secured to the framework 32 and electrically coupled to the materials 34 and 36. The control device 38 includes electronic circuitry, for example, a memory, a control logic that is capable of controlling and altering the conductance between the materials 34 and 36.

The voltage potential created between the materials 34 and 36 provides the power for operating the system as well as produces the current flow through the conducting fluid and the system. In one aspect, the system operates in direct current mode. In an alternative aspect, the system controls the direction of the current so that the direction of current is reversed in a cyclic manner, similar to alternating current. As the system reaches the conducting fluid or the electrolyte, where the fluid or electrolyte component is provided by a physiological fluid, e.g., stomach acid, the path for current flow between the materials 34 and 36 is completed external to the system 230; the current path through the system 30 is controlled by the control device 38. Completion of the current path allows for the current to flow, through conductive communication through the stomach, and in turn to a receiver, not shown, the receiver capable of detecting the presence of the current signature containing information and further recognize that the system 30 has been activated and the desired event is occurring or has occurred.

In one aspect, the two materials 34 and 36 are similar in function to the two electrodes needed for a direct current power source, such as a battery. The conducting liquid acts as the electrolyte needed to complete the power source.

The completed power source described is defined by the physical chemical reaction between the materials 34 and 36 of the system 30 and the surrounding fluids of the body. The completed power source may be viewed as a power source that exploits reverse electrolysis in an ionic or a conductive solution such as gastric fluid, blood, or other bodily fluids and some tissues. Additionally, the environment may be something other than a body and the liquid may be any conducting liquid. For example, the conducting fluid may be salt water or a metallic based paint.

Referring again to FIG. 1, the materials 34 and 36 provide the voltage potential to activate the control device 38. Once the control device 38 is activated or powered up, the control device 38 can alter conductance between the materials 34 and 36 in a unique or desired manner. By altering the conductance between materials 34 and 236, the control device 38 is capable of controlling the magnitude of the current through the conducting liquid that surrounds the system 30. This produces a unique current signature that can be detected and measured by a receiver (not shown), which can be positioned internal, over or external to the body.

As described above, in various aspects, the event indicator may further include a current path extender such as a membrane which, for example, produces a virtual dipole length between the pair of transmission elements that is larger than the actual dipole length. As illustrated in FIG. 1, the current path extender or “skirt”, shown in portion at 35 and 37, respectively, may be associated with, e.g., secured to, the framework 32. Various shapes and configurations for the skirt are contemplated as within the scope of the present invention. For example, the system 30 may be surrounded entirely or partially by the skirt and the skirt may be positioned along a central axis of the system 30 or off-center relative to a central axis. Thus, the scope of the present invention as claimed herein is not limited by the shape or size of the skirt. Furthermore, in other aspects, the materials 34 and 36 may be separated by one skirt that is positioned in any defined region between the materials 34 and 36.

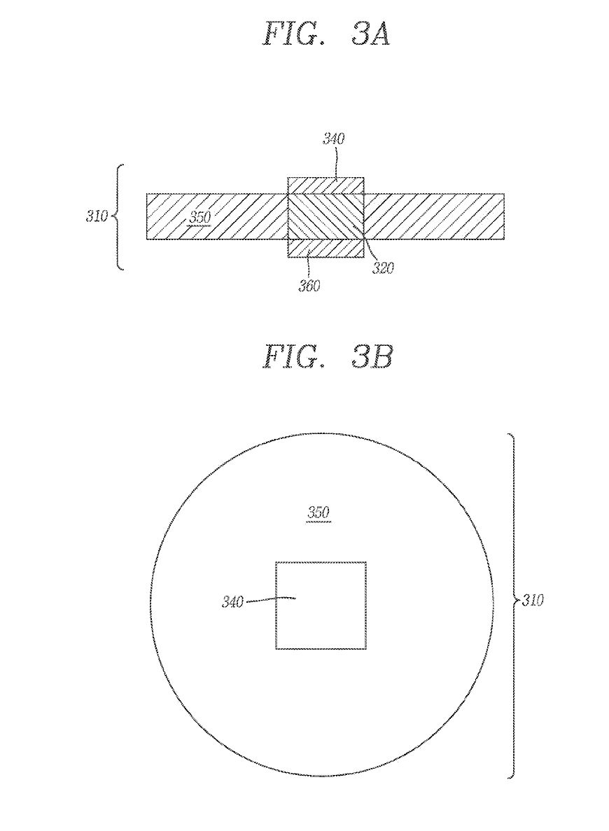

FIG. 3A provides a view of an aspect of an IEM of interest which has a current path extender in the form of a membrane that extends beyond the outer edges of the signal transmission elements to provide a virtual dipole having a length that is longer than the actual dipole between the signal transmission elements. As shown in FIG. 3A, event indicator 310 includes integrated circuit 320, having a first electrochemical material 340 (which may comprise two distinct material layers) and a second electrochemical material 360. Also shown is disc-shaped membrane 350. FIG. 3B provides an overhead view of the event indicator shown in FIG. 3A, showing the disc shape of first electrochemical material 340 and the positioning of the first electrochemical material in the center of disc-shaped membrane 350. The distance that the edge of the membrane may extend beyond the edge of electrodes may vary, and in certain aspects is 0.05 mm or more, e.g., 0.1 mm or more, including 1.0 mm or more, such as 5.0 mm or more and including 10 mm or more, where the distance may not exceed 100 mm in certain aspects.

As can be seen in the aspect depicted in FIGS. 3A & 3B, the first and second electrochemical materials may have any convenient shape, e.g., square, disc, etc. The disc-shaped membrane 350 is a planar disc structure, where the edge of the membrane extends beyond the edge of the first and second electrochemical materials. In the depicted aspect, the radius of the membrane is longer than the radius of the first and second electrochemical materials, e.g., by 1 mm or more, such as by 10 mm or more. Membranes may have “two-dimensional” or “three-dimensional” configurations, as desired. Membrane configurations of interest are further described in PCT Application Serial No. PCT/US2008/077753 published as WO2009/042812, PCT Application Serial No. PCT/US2010/020142 published as WO2010/080765 as well as PCT Application Serial No. PCT/US2010/032590 published as WO2010/129288; the disclosures of which are herein incorporated by reference. The membrane may be fabricated from a number of different materials, where the membrane may be made of a single material or be a composite of two or more different types of materials, as developed in greater detail below. Where desired, the membrane may be made up partially or completely of a highly-swellable polymeric film, e.g., as described in U.S. Provisional Application Ser. No. 61/758,030 filed on Jan. 29, 2013; the disclosure of which is herein incorporated by reference. In such instances, the highly-swellable polymeric film is associated with the support and is configured as a signal amplification that increases a length of a current path between the first and second materials. In certain instances, the membrane will have a mechanical strength sufficient to withstand the mechanical forces typical of the gastrointestinal (GI) tract without folding onto itself and losing its shape. This desired mechanical strength may be chosen to last for at least the duration of the communication, which may be 1 second or longer, such as at least 1 minute or longer, up to 6 hours or longer. In certain aspects, the desired mechanical strength is selected to last least for a period of time ranging from 1 to 30 minutes. The desired mechanical strength can be achieved by proper selection of polymer and/or fillers, or mechanical design (e.g., lamination of multiple layers, or curvature of the amplifier surface) to increase the mechanical strength of the final structure. Membranes of the invention are ones that are electrically insulating. As such, the materials from which the membranes are fabricated are electrically insulating materials. A given material is electrically insulating if it has a resistivity that is two times or greater than the medium in which the device operates, e.g., stomach fluid, such as ten times or greater, including 100 times or greater than the medium in which the device operates.

Referring now to FIG. 4, in another aspect of an ingestible device is shown in more detail as system 440. The system 440 includes a framework 442. The framework 442 is similar to the framework 32 of FIG. 1. In this aspect of the system 440, a digestible or dissolvable material 444 is deposited on a portion of one side of the framework 442. At a different portion of the same side of the framework 442, another digestible material 446 is deposited, such that materials 444 and 446 are dissimilar. More specifically, material 444 and 446 are selected such that they form a voltage potential difference when in contact with a conducting liquid, such as body fluids. Thus, when the system 440 is in contact with and/or partially in contact with the conducting liquid, then a current path, an example is shown in FIG. 2, is formed through the conducting liquid between material 444 and 446. A control device 448 is secured to the framework 442 and electrically coupled to the materials 444 and 446. The control device 448 includes electronic circuitry that is capable of controlling part of the conductance path between the materials 444 and 446. The materials 444 and 446 are separated by a non-conducting skirt 449. Various examples of the skirt 449 are disclosed in PCT Application No. PCT/US2010/032590 published as WO2010/129288; and PCT application Ser. No. PCT/US2008/077753 published as WO2009/042812; the entire disclosure of each is incorporated herein by reference. Once the control device 448 is activated or powered up, the control device 448 can alter conductance between the materials 444 and 446. Thus, the control device 448 is capable of controlling the magnitude of the current through the conducting liquid that surrounds the system 440. As indicated above with respect to system 30, a unique current signature that is associated with the system 440 can be detected by a receiver (not shown) to mark the activation of the system 440. In order to increase the “length” of the current path the size of the skirt 449 is altered. The longer the current path, the easier it may be for the receiver to detect the current.

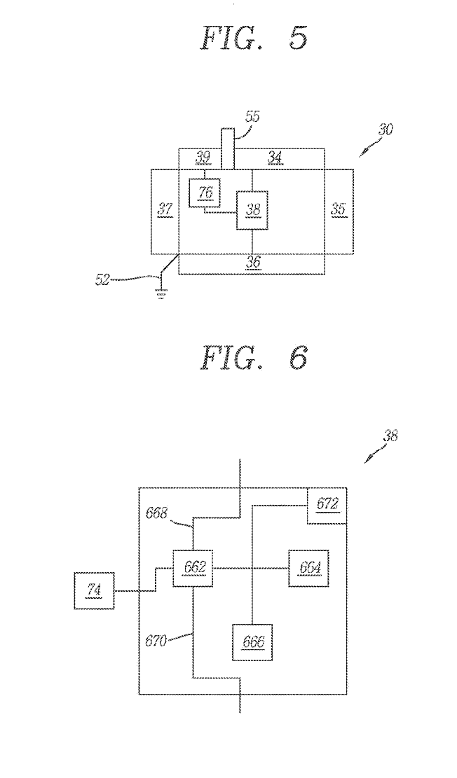

Referring now to FIG. 2, the system 30 of FIG. 1 is shown in an activated state and in contact with conducting liquid. The system 30 is grounded through ground contact 52. The system 30 also includes a sensor module 74, which is described in greater detail with respect to FIG. 6. Ion or current paths 550 form between material 34 to material 36 through the conducting fluid in contact with the system 30. The voltage potential created between the material 34 and 36 is created through chemical reactions between materials 34/36 and the conducting fluid. FIG. 2A provides an exploded view of the surface of material 34. The surface of the material 34 is not planar, but rather an irregular surface 54 as shown. The irregular surface 54 increases the surface area of the material and, hence, the area that comes in contact with the conducting fluid. In one aspect, at the surface of the material 34, there is chemical reaction between the material 34 and the surrounding conducting fluid such that mass is released into the conducting fluid. The term “mass” as used herein refers to protons and neutrons that form a substance. One example includes where the material is CuCI and when in contact with the conducting fluid, CuCI becomes Cu (solid) and CI in solution. The flow of ions into the conduction fluid is depicted by the ion paths 50. In a similar manner, there is a chemical reaction between the material 36 and the surrounding conducting fluid and ions are captured by the material 36. The release of ions at the material 34 and capture of ion by the material 36 is collectively referred to as the ionic exchange. The rate of ionic exchange and, hence the ionic emission rate or flow, is controlled by the control device 38. The control device 38 can increase or decrease the rate of ion flow by altering the conductance, which alters the impedance, between the materials 34 and 36. Through controlling the ion exchange, the system 30 can encode information in the ionic exchange process. Thus, the system 30 uses ionic emission to encode information in the ionic exchange.

The control device 38 can vary the duration of a fixed ionic exchange rate or current flow magnitude while keeping the rate or magnitude near constant, similar to when the frequency is modulated and the amplitude is constant. Also, the control device 38 can vary the level of the ionic exchange rate or the magnitude of the current flow while keeping the duration near constant. Thus, using various combinations of changes in duration and altering the rate or magnitude, the control device 38 encodes information in the current flow or the ionic exchange. For example, the control device 38 may use, but is not limited to any of the following techniques namely, Binary Phase-Shift Keying (PSK), Frequency modulation, Amplitude modulation, on-off keying, and PSK with on-off keying.

As indicated above, the various aspects disclosed herein, such as systems 30 and 440 of FIGS. 1 and 4, respectively, include electronic components as part of the control device 38 or the control device 448. Components that may be present include but are not limited to: logic and/or memory elements, an integrated circuit, an inductor, a resistor, and sensors for measuring various parameters. Each component may be secured to the framework and/or to another component. The components on the surface of the support may be laid out in any convenient configuration. Where two or more components are present on the surface of the solid support, interconnects may be provided.

As indicated above, the system, such as system 30 and 440, control the conductance between the dissimilar materials and, hence, the rate of ionic exchange or the current flow. Through altering the conductance in a specific manner the system is capable of encoding information in the ionic exchange and the current signature. The ionic exchange or the current signature is used to uniquely identify the specific system. Additionally, the systems 30 and 440 are capable of producing various different unique exchanges or signatures and, thus, provide additional information. For example, a second current signature based on a second conductance alteration pattern may be used to provide additional information, which information may be related to the physical environment. To further illustrate, a first current signature may be a very low current state that maintains an oscillator on the chip and a second current signature may be a current state at least a factor of ten higher than the current state associated with the first current signature.

Referring now to FIG. 6, a block diagram representation of the control device 38 is shown. The device 38 includes a control module 662, a counter or clock 664, and a memory 666. Additionally, the device 38 is shown to include a sensor module 672 as well as the sensor module 74, which was referenced in FIG. 2. The control module 662 has an input 668 electrically coupled to the material 34 and an output 670 electrically coupled to the material 36. The control module 662, the clock 664, the memory 666, and the sensor modules 672/274 also have power inputs (some not shown). The power for each of these components is supplied by the voltage potential produced by the chemical reaction between materials 34 and 36 and the conducting fluid, when the system 30 is in contact with the conducting fluid. The control module 662 controls the conductance through logic that alters the overall impedance of the system 30. The control module 662 is electrically coupled to the clock 664. The clock 64 provides a clock cycle to the control module 662. Based upon the programmed characteristics of the control module 662, when a set number of clock cycles have passed, the control module 662 alters the conductance characteristics between materials 34 and 36. This cycle is repeated and thereby the control device 38 produces a unique current signature characteristic. The control module 662 is also electrically coupled to the memory 666. Both the clock 664 and the memory 666 are powered by the voltage potential created between the materials 34 and 36.

The control module 662 is also electrically coupled to and in communication with the sensor modules 672 and 274. In the aspect shown, the sensor module 672 is part of the control device 38 and the sensor module 74 is a separate component. In alternative aspects, either of the sensor modules, 672 and 74, can be used without the other and the scope of the present invention is not limited by the structural or functional location of the sensor modules 672 or 74. Additionally, any component of the system 30 may be functionally or structurally moved, combined, or repositioned without limiting the scope of the present invention as claimed. Thus, it is possible to have one single structure, for example a processor, which is designed to perform the functions of all of the following modules: the control module 662, the clock 664, the memory 666, and the sensor module 672 or 274. On the other hand, it is also within the scope of the present invention to have each of these functional components located in independent structures that are linked electrically and able to communicate.

Referring again to FIG. 6, the sensor modules 672 or 274 can include any of the following sensors: temperature, pressure, pH level, and conductivity. In one aspect, the sensor modules 672 or 74 gather information from the environment and communicate the analog information to the control module 662. The control module then converts the analog information to digital information and the digital information is encoded in the current flow or the rate of the transfer of mass that produces the ionic flow. In another aspect, the sensor modules 672 or 74 gather information from the environment and convert the analog information to digital information and then communicate the digital information to control module 662.

In the aspect shown in FIG. 2, the sensor module 74 is shown as being electrically coupled to the materials 34 and 36 as well as the control device 38. In another aspect, as shown in FIG. 6, the sensor module 74 is electrically coupled to the control device 38 at connection 678. The connection 678 acts as both a source for power supply to the sensor module 74 and as a communication channel between the sensor module 74 and the control device 38.

Referring now to FIG. 5, the system 30 includes a pH sensor module 76 connected to a material 39, which is selected in accordance with the specific type of sensing function being performed. The pH sensor module 76 is also connected to the control device 38. The material 39 is electrically isolated from the material 34 by a non-conductive barrier 55. In one aspect, the material 39 is platinum. In operation, the pH sensor module 76 uses the voltage potential difference between the materials 234/236. The pH sensor module 76 measures the voltage potential difference between the material 34 and the material 39 and records that value for later comparison. The pH sensor module 76 also measures the voltage potential difference between the material 39 and the material 36 and records that value for later comparison. The pH sensor module 76 calculates the pH level of the surrounding environment using the voltage potential values. The pH sensor module 76 provides that information to the control device 38. The control device 38 varies the rate of the transfer of mass that produces the ionic transfer and the current flow to encode the information relevant to the pH level in the ionic transfer, which can be detected by a receiver (not shown). Thus, the system 30 can determine and provide the information related to the pH level to a source external to the environment.

As indicated above, the control device 38 can be programmed in advance to output a pre-defined current signature. In another aspect, the system can include a receiver system that can receive programming information when the system is activated. In another aspect, not shown, the switch 664 and the memory 666 can be combined into one device.

In addition to the above components, the system 30 may also include one or other electronic components. Electrical components of interest include, but are not limited to: additional logic and/or memory elements, e.g., in the form of an integrated circuit; a power regulation device, e.g., battery, fuel cell or capacitor; a sensor, a stimulator, etc.; a signal transmission element, e.g., in the form of an antenna, electrode, coil, etc.; a passive element, e.g., an inductor, resistor, etc.

As mentioned above, highly-swellable polymeric films may be associated with an ingestible event marker in a number of different, non-mutually exclusive ways. For example, the film may cover a portion of at least one of the first or second dissimilar materials. As such, the film may cover a portion or all of the cathode material. Alternatively or in addition, the film may cover a portion or all of the anode material. In such instances, the film may cover 10% or more, 20% or more, 25% percent or more, 50% or more, including 75% percent or more, e.g., 90% or more, of the cathode and/or anode material, including all of the cathode material or anode material.

An edible adhesive composition, e.g., as described herein, is associated with the highly-swellable polymeric film and the ingestible component in a manner that mediates adherence of the film and the ingestible component to each other.

The adhesive may cover a portion of all of one or both of the surfaces of the film and ingestible component that are facing each other when adhered to each other by the adhesive. As such, the adhesive may cover a portion of or the entire surface of the film that faces the ingestible component in the adhered state. Accordingly, the adhesive may 10% or more, 20% or more, 30% or more, 40% or more, 50% or more, 60% or more, 70% or more, 80% or more, 90% or more, including 95% or more of the entire surface of the film that faces the ingestible component in the adhered state. Similarly, the adhesive may cover a portion of or the entire surface of the ingestible component that faces the film in the adhered state. Accordingly, the adhesive may 10% or more, 20% or more, 30% or more, 40% or more, 50% or more, 60% or more, 70% or more, 80% or more, 90% or more, including 95% or more of the entire surface of the ingestible component that faces the film in the adhered state.

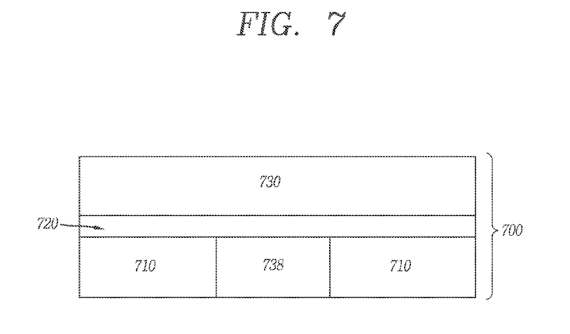

An example of an IEM which includes an adhesive as described above is depicted in FIG. 7. As shown in FIG. 7, IEM 700 includes a control element 738 surround by a current amplifier 710. Present on the upper surface of control element 738 is adhesive 720. Stably associated with the control element 738 and current amplifier 710 by adhesive layer 720 is highly-swellable polymer film 730. While the thickness of adhesive layer 720 may vary, in some instances the thickness ranges from 5 to 50 microns.

### Other Minimally Dimensioned Components

Aspects of the invention further include compositions that are not necessarily ingestible, wherein such compositions may include a highly-swellable polymeric film adhered to a minimally dimensioned component by an adhesive, e.g., as described herein. Such compositions may include a highly-swellable polymeric film (e.g., as described in greater detail above) physically associated with a minimally dimensioned component. While the minimally dimensioned component may vary, e.g., as described above, in some instances the minimally dimensioned component is a micro-battery. Micro-batteries of interest may include “all-solid” batteries, and may include components of a battery, such as current collectors, positive and negative electrodes, an electrolyte, in a minimally dimensioned structure, e.g., as described above. In some instances, micro-batteries of interest are thin films, which may be obtained by deposition, such as by physical vapor deposition (PVD) or chemical vapor deposition (CVD). The micro-battery may take a variety of different configurations, such as but not limited to: a chip configuration, a cylinder configuration, a spherical configuration, a disc configuration, etc., where a particular configuration may be selected based on intended application, method of manufacture, etc. In some instances, the micro-battery is dimensioned to have a width ranging from about 0.05 mm to about 1 mm, such as from about 0.1 mm to about 0.2 mm; a length ranging from about 0.05 mm to about 1 mm, such as from about 0.1 mm to about 0.2 mm and a height ranging from about 0.1 mm to about 1 mm, such as from about 0.05 mm to about 0.3 mm, including from about 0.1 mm to about 0.2 mm. In certain instances, the micro-battery is 1 mm³ or smaller, such as 0.1 mm³ or smaller, including 0.2 mm³ or smaller. In such instances the film may serve to enhance stability of the component, e.g., by improving shelf-life, etc., such as described above.

### Manufacturing Methods

Also provided are methods of manufacturing edible adhesive compositions, as well as ingestible compositions that include the same. The adhesive compositions may be prepared as films, which may be free-standing or present on a support. For example, solvent casting fabrication methods may be employed in which a liquid composition of the adhesive components is prepared by combining the components and a suitable solvent(s). Solvents of interest include, but are not limited to: water and aqueous solvents which include one or more solutes (e.g., salts); organic solvents, e.g., alcohols, such as ethanol, propanol, isopropanol, methanol, butanol, etc.; polyols, e.g., propylene glycol, glycerin, butylene glycol, ethoxydiglycol, polyethylene glycol, methyl or ethyl ethers of diglycols, cyclic polyols, ethoxylated or propoxylated glycols; and other organic solvents, e.g., Heptane, Isobutyl acetate, Butyl acetate, Methylethylketone, tert-Butylmethyl ether, Methylisobutylketone, Dimethyl sulfoxide, Pentane, Toluene, Trichloroethylene, and Xylene; etc. The liquid composition may be prepared at any convenient temperature, e.g., 15 to 45° C., using any convenient protocol, e.g., stirring, etc. Following preparation of the liquid composition, the liquid composition may be cast on a suitable support, such as a release liner, etc., following which the solvent may be separated from the remainder of the liquid composition to produce the adhesive composition which is ready for use. Solvent separation may be achieved a number of different ways, e.g., via evaporation, which may occur at room temperature or elevated temperatures, such that the temperature may range in some instances from 25 to 100° C., such as 45 to 75° C.

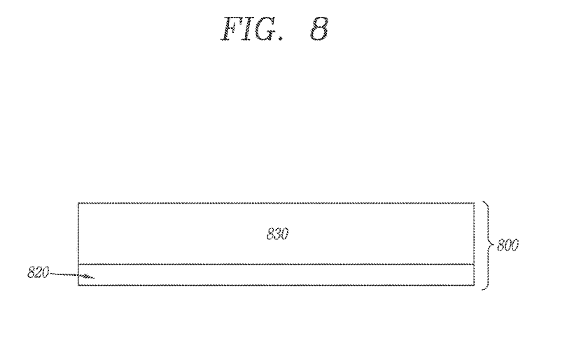

FIG. 8 provides a side view of an adhesive composition that has been cast onto a release liner, e.g., as described above. In FIG. 8, solvent cast adhesive composition 800 includes an adhesive film 830 present on support 820. Support 820 may be fabricated from any suitable material, such as but not limited to: polyesters, such as polyethylene terephthalate, polypropylene, and the like. In certain embodiments, the release liner includes a coated substrate, which, for example, may be prepared by treating one side of polyethylene-coated wood free paper, polyolefin-coated glassine paper, a polyethylene terephthalate (polyester) film, a polypropylene film, or the like with a silicone treatment. In certain instances, the release liner includes a polyester film with a silicone treatment. While the release liner thickness may vary, in some instances the thickness of the release liner ranges from 1 to 10 mils, e.g., 1 to 5 mils, such as 2 to 3 mils.

For ingestible compositions that include a highly-swellable polymeric film and an ingestible component which are adhered to each other by an edible adhesive, e.g., as described herein, aspects of the methods include combining an ingestible component (which may or may not include a device, such as an IEM) and a highly-swellable polymeric film, e.g., as described above, with an edible adhesive composition in a manner sufficient to produce a desired ingestible composition in which the film is adhered to the ingestible component by the adhesive. Any convenient manufacturing protocol may be employed, where protocols of interest include both manual and automated protocols, as well as protocols that include both manual and automated steps. Protocols of interest that find use in various aspects of the fabrication methods described herein include lamination, molding, pressing, extrusion, stamping, coating (such as spray coating and dipping), gluing, etc. In some instances, fabrication protocols as described in PCT application serial nos. PCT/US2010/020142 published as WO 2010/080765; PCT/US2006/016370 published as WO 2006/116718 and PCT/US2008/077753 published as WO2009/042812 (the disclosures of which applications are herein incorporated by reference); are employed.

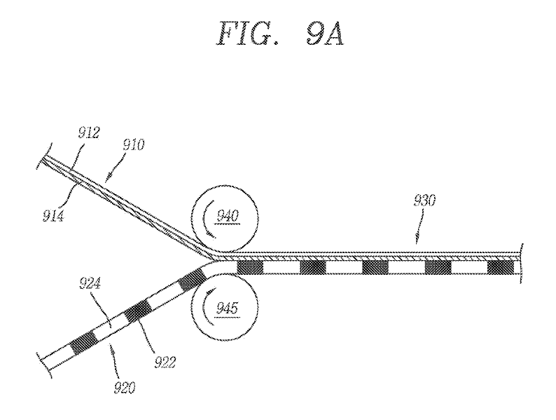

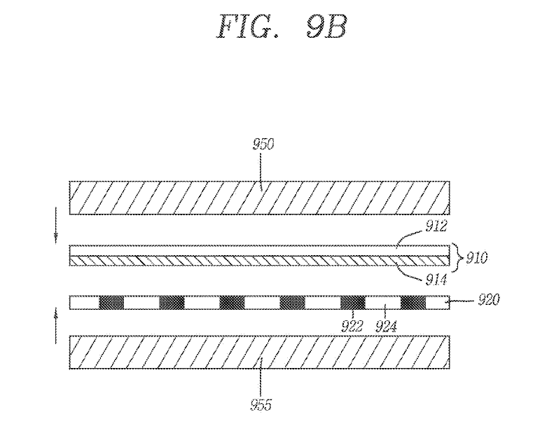

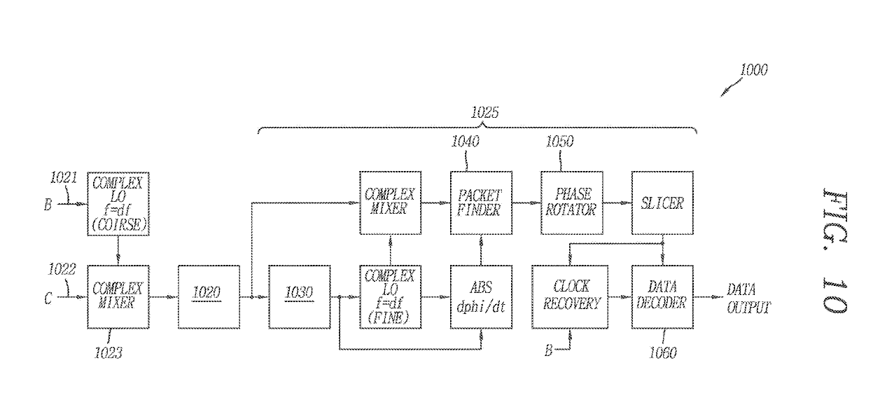

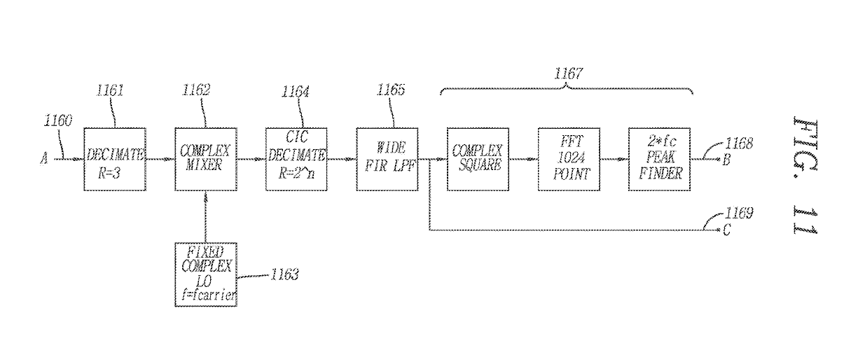

Aspects of the fabrication protocols include stably associating the ingestible component with a highly-swellable polymeric film component using an edible adhesive composition, e.g., as described above. By “stably associating” is meant that the ingestible component and highly-swellable polymeric film component do not separate from each other, at least until desired during intended use, e.g., upon administration to a subject in need thereof, such as by ingestion. Any convenient approach for stably associating the ingestible component and the highly-swellable polymeric film component using an edible adhesive may be employed.

Where an ingestible event marker having one of its dissimilar materials covered by a highly-swellable polymeric film is desired, e.g., as illustrated in FIG. 7, a protocol in which a pre-fabricated adhesive film, e.g., prepared as described above, may be employed. FIGS. 9A and 9B provide illustrations of two different embodiments of such protocols. In FIG. 9A, a continuous roller lamination fabrication process is depicted. In FIG. 9A, a film sheet 910 is laminated to IEM sheet 920 to produce a laminated IEM sheet 930 which can then be singulated into individual IEMs, e.g., with a punch device. Film sheet is made up of swellable film 912 and edible adhesive 914. IEM sheet 920 is made up of individual IEM circuitry components 922 present in signal amplifier (i.e., skirt) material 924. The IEM and film sheets, 920 and 910 respectively, are brought together between rollers 940 and 945 with heat and/or pressure to produce laminated IEM sheet 930, which is made up of the film sheet 910 adhered to the surface of IEM sheet 920 by adhesive 914. FIG. 9B depicts a press lamination fabrication protocol. In FIG. 9B film sheet 910 and IEM sheet 920 are positioned between platens 950 and 955. During lamination, platens 950 and 955 are moved toward each other in the direction of the arrows to apply pressure (and optionally heat) to the IEM and film sheets to produce a laminated IEM sheet (not shown), which can subsequently be singulated into individual IEMs, e.g., as described above.

In yet another fabrication protocol of interest, a coating process may be employed. For example, a premade ingestible component in the form of a tablet may be provided, e.g., as described in in PCT application serial nos. PCT/US2010/020142 published as WO 2010/080765; PCT/US2006/016370 published as WO 2006/116718 and PCT/US2008/077753 published as WO2009/042812; the disclosures of which applications are herein incorporated by reference. This premade ingestible component may then be spray coated with a liquid precursor composition of the adhesive (e.g., as described above). Following spray coating, a highly-swellable polymeric film may be adhered to the ingestible component via the adhesive to produce the desired product.

### Systems

Also provided are systems that include an ingestible device, e.g., an IEM, and a detection component, e.g., in the form of a receiver. Receivers of interest are those configured to detect, e.g., receive, a communication from an IEM. The signal detection component may vary significantly depending on the nature of the communication that is generated by the ingestible device. As such, the receiver may be configured to receive a variety of different types of signals, including but not limited to: RF signals, magnetic signals, conductive (near field) signals, acoustic signals, etc. In certain aspects, the receiver is configured to receive a signal conductively from an IEM, such that the two components use the body of the patient as a communication medium. As such, communication that is transferred between event indicator and the receiver travels through the body, and requires the body as the conduction medium. The IEM communication may be transmitted through and received from the skin and other body tissues of the subject body in the form of electrical alternating current (a.c.) voltage signals that are conducted through the body tissues. This communication protocol has the advantage that the receivers may be adaptably arranged at any desired location on the body of the subject, whereby the receivers are automatically connected to the required electrical conductor for achieving the signal transmission, i.e., the signal transmission is carried out through the electrical conductor provided by the skin and other body tissues of the subject.

The receivers of interest include external, semi-implantable, and implantable receivers. In external aspects, the receiver is ex vivo, by which is meant that the receiver is present outside of the body during use. Examples include wearable patches, e.g., adhesive patches, torso bands, wrist(s) or arm bands, jewelry, apparel, mobile devices such as phones, attachments to mobile devices, etc. Where the receiver is implanted, the receiver is in vivo. Examples include cardiac can and leads, under-the-skin implants, etc. Semi-implantable devices include those designed to be partially implanted under the skin.

In certain aspects, the receiver may be configured to provide data associated with a received signal to a location external to said subject. For example, the receiver may be configured to provide data to an external data receiver, e.g., which may be in the form of a monitor (such as a bedside monitor), a computer, a personal digital assistant (PDA), phone, messaging device, smart phone, etc. The receiver may be configured to retransmit data of a received communication to the location external to said subject. Alternatively, the receiver may be configured to be interrogated by an external interrogation device to provide data of a received signal to an external location.

Receivers may be configured variously, e.g., with various signal receiving elements, such as electrodes, various integrated circuit components, one or more power components (such as power receivers or batteries), signal transmission components, housing components, etc.

In one aspect, for example, the receiver includes one or more of: a high power-low power module; an intermediary module; a power supply module configured to activate and deactivate one or more power supplies to a high power processing block; a serial peripheral interface bus connecting master and slave blocks; and a multi-purpose connector, as further described in PCT Application Serial No. PCT/US2009/068128 published as WO2010/075115, infra.

Receivers of interest include, but are not limited to, those receivers disclosed in: PCT Application Serial No. PCT/US2006/016370 published as WO 2006/116718; PCT Application Serial No. PCT/US2008/52845 published as WO 2008/095183; PCT Application Serial No. PCT/US2007/024225 published as WO 2008/063626; PCT Application Serial No. PCT/US2008/085048 published as WO 2009/070773; PCT Application Serial No. PCT/US2009/068128 published as WO2010/075115; and PCT Application Serial No. PCT/US2012/047076 filed on Jul. 21, 2012; the disclosures of which applications (and particularly receiver components thereof) are herein incorporated by reference.

In certain embodiments, the signal receiver includes a set of two or more, such as two or three, electrodes that provide for dual functions of signal receiving and sensing. For example, in addition to receiving signal, the electrodes can also serve additional sensing functions. In certain embodiments, the electrodes are used to generate electrocardiogram data. From that data, there are many kinds of processing that can be done, e.g., to detect various cardiac events, such as tachycardia, fibrillations, heart rate, etc. The obtained electrocardiogram data can be used to titrate medications, or be used for alerts when an important change or significant abnormality in the heart rate or rhythm is detected. This data is also helpful in certain embodiments for monitoring heart rate in patients who do not have pacemakers or as an alternative to patients who might normally require a Holter monitor or a Cardiac Event Monitor, portable devices for continuously monitoring the electrical activity of the heart for twenty-four hours or other devices. An extended recording period is useful for observing occasional cardiac arrthymias that are difficult to identify in shorter time periods.

In some instances, two or more different demodulation protocols may be employed to decode a given received signal. In some instances, both a coherent demodulation protocol and a differential coherent demodulation protocol may be employed. FIG. 10 provides a functional block diagram of how a receiver may implement a coherent demodulation protocol, according to one aspect of the invention. It should be noted that only a portion of the receiver is shown in FIG. 10. FIG. 10 illustrates the process of mixing the signal down to baseband once the carrier frequency (and carrier signal mixed down to carrier offset) is determined. A carrier signal 1021 is mixed with a second carrier signal 1022 at mixer 1023. A narrow low-pass filter 1020 is applied of appropriate bandwidth to reduce the effect of out-of-bound noise. Demodulation occurs at functional blocks 1025 in accordance with the coherent demodulation scheme of the present invention. The unwrapped phase 1030 of the complex signal is determined. An optional third mixer stage, in which the phase evolution is used to estimate the frequency differential between the calculated and real carrier frequency can be applied. The structure of the packet is then leveraged to determine the beginning of the coding region of the BPSK signal at block 1040. Mainly, the presence of the sync header, which appears as an FM porch in the amplitude signal of the complex demodulated signal is used to determine the starting bounds of the packet. Once the starting point of the packet is determined the signal is rotated at block 1050 on the IQ plane and standard bit identification and eventually decoded at block 1060.

In addition to demodulation, the trans-body communication module may include a forward error correction module, which module provides additional gain to combat interference from other unwanted signals and noise. Forward error correction functional modules of interest include those described in PCT Application Serial No. PCT/US2007/024225 published as WO 2008/063626; the disclosure of which application is herein incorporated by reference. In some instances, the forward error correction module may employ any convenient protocol, such as Reed-Solomon, Golay, Hamming, BCH, and Turbo protocols to identify and correct (within bounds) decoding errors.

Receivers of the invention may further employ a beacon functionality module. In various aspects, a beacon switching module may employ one or more of the following: a beacon wakeup module, a beacon signal module, a wave/frequency module, a multiple frequency module, and a modulated signal module.

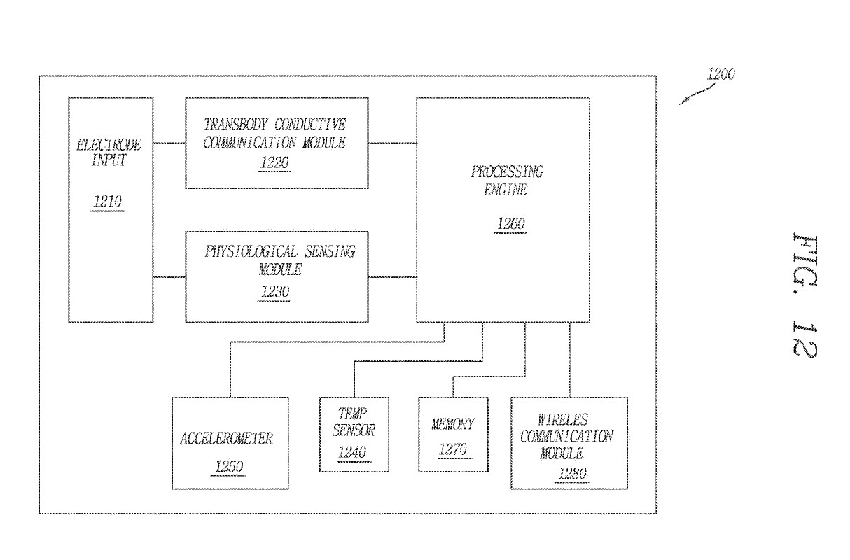

A view of a beacon module is provided in the functional block diagram shown in 11. The scheme outlined in FIG. 11 outlines one technique for identifying a valid beacon. The incoming signal 1160 represents the signals received by electrodes, bandpass filtered (such as from 10 KHz to 34 KHz) by a high frequency signaling chain (which encompasses the carrier frequency), and converted from analog to digital. The signal 1160 is then decimated at block 1161 and mixed at the nominal drive frequency (such as, 12.5 KHz, 20 KHz, etc.) at mixer 1162. The resulting signal is decimated at block 1164 and low-pass filtered (such as 5 KHz BW) at block 1165 to produce the carrier signal mixed down to carrier offset—signal 1169. Signal 1169 is further processed by blocks 1167 (fast Fourier transform and then detection of two strongest peaks) to provide the true carrier frequency signal 1168. This protocol allows for accurate determination of the carrier frequency of the transmitted beacon. Further examples of beacon functionality modules are described in PCT Application Serial No. PCT/US2008/085048 published as WO 2009/070773; the disclosure of which application is herein incorporated by reference.

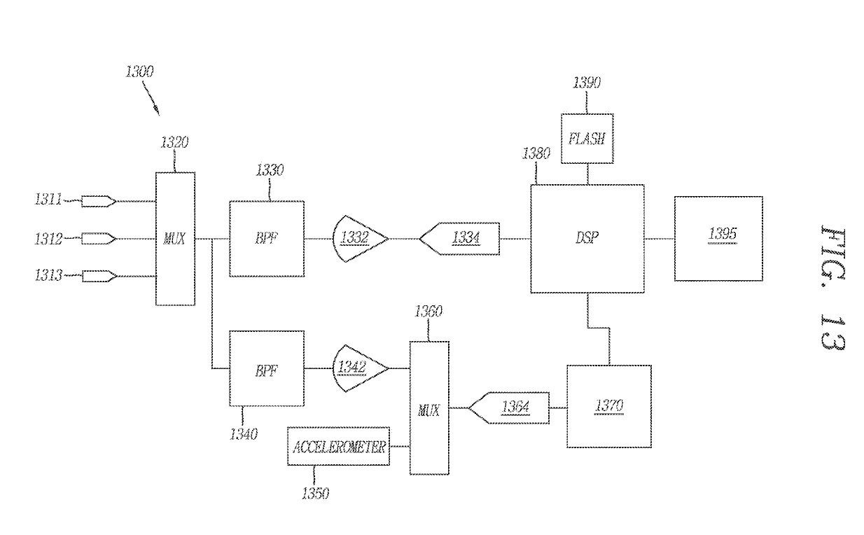

FIG. 12 provides a block functional diagram of an integrated circuit component of a signal receiver according to an aspect of the invention. In FIG. 12, receiver 1200 includes electrode input 1210. Electrically coupled to the electrode input 1210 are trans-body conductive communication module 1220 and physiological sensing module 1230. In one aspect, trans-body conductive communication module 1220 is implemented as a high frequency (HF) signal chain and physiological sensing module 1230 is implemented as a low frequency (LF) signal chain. Also shown are CMOS temperature sensing module 1240 (for detecting ambient temperature) and a three-axis accelerometer 1250. Receiver 1200 also includes a processing engine 1260 (for example, a microcontroller and digital signal processor), non-volatile memory 1270 (for data storage) and wireless communication module 1280 (for data transmission to another device, for example in a data upload action).

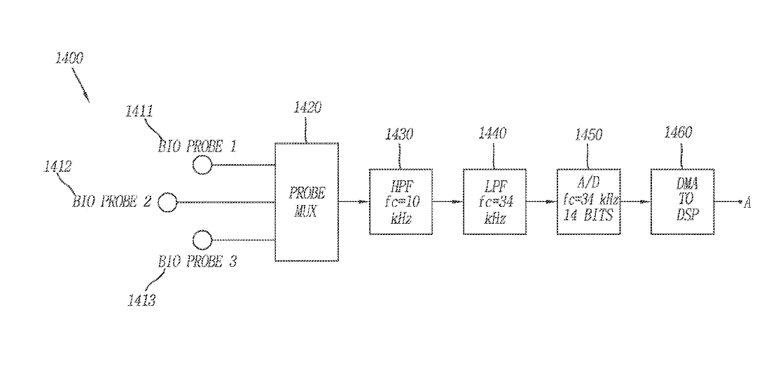

FIG. 13 provides a more detailed block diagram of a circuit configured to implement the block functional diagram of the receiver depicted in FIG. 12, according to one aspect of the invention. In FIG. 13, receiver 1300 includes electrodes e1 , e2 and e3 (1311, 1312 and 1313) which, for example, receive the conductively transmitted signals by an IEM and/or sense physiological parameters or biomarkers of interest. The signals received by the electrodes 1311, 1312, and 1313 are multiplexed by multiplexer 1320 which is electrically coupled to the electrodes.

Multiplexer 1320 is electrically coupled to both high band pass filter 1330 and low band pass filter 1340. The high and low frequency signal chains provide for programmable gain to cover the desired level or range. In this specific aspect, high band pass filter 1330 passes frequencies in the 10 KHz to 34 KHz band while filtering out noise from out-of-band frequencies. This high frequency band may vary, and may include, for example, a range of 3 KHz to 300 KHz. The passing frequencies are then amplified by amplifier 1332 before being converted into a digital signal by converter 834 for input into high power processor 1380 (shown as a DSP) which is electrically coupled to the high frequency signal chain.

Low band pass filter 1340 is shown passing lower frequencies in the range of 0.5 Hz to 150 Hz while filtering out out-of-band frequencies. The frequency band may vary, and may include, for example, frequencies less than 300 Hz, such as less than 200 Hz, including less than 150 Hz. The passing frequency signals are amplified by amplifier 1342. Also shown is accelerometer 1350 electrically coupled to second multiplexer 1360. Multiplexer 1360 multiplexes the signals from the accelerometer with the amplified signals from amplifier 1342. The multiplexed signals are then converted to digital signals by converter 1364 which is also electrically coupled to low power processor 1370.

In one aspect, a digital accelerometer (such as one manufactured by Analog Devices), may be implemented in place of accelerometer 1350. Various advantages may be achieved by using a digital accelerometer. For example, because the signals the digital accelerometer would produce signals already in digital format, the digital accelerometer could bypass converter 1364 and electrically couple to the low power microcontroller 1370—in which case multiplexer 1360 would no longer be required. Also, the digital signal may be configured to turn itself on when detecting motion, further conserving power. In addition, continuous step counting may be implemented. The digital accelerometer may include a FIFO buffer to help control the flow of data sent to the low power processor 1370. For instance, data may be buffered in the FIFO until full, at which time the processor may be triggered to turn awaken from an idle state and receive the data.

Low power processor 1370 may be, for example, an MSP430 microcontroller from Texas Instruments. Low power processor 1370 of receiver 800 maintains the idle state, which as stated earlier, requires minimal current draw—e.g., 10 μA or less, or 1 μA or less.

High power processor 1380 may be, for example, a VC5509 digital signal process from Texas Instruments. The high power processor 1380 performs the signal processing actions during the active state. These actions, as stated earlier, require larger amounts of current than the idle state—e.g., currents of 30 μA or more, such as 50 μA or more—and may include, for example, actions such as scanning for conductively transmitted signals, processing conductively transmitted signals when received, obtaining and/or processing physiological data, etc.

The receiver may include a hardware accelerator module to process data signals. The hardware accelerator module may be implemented instead of, for example, a DSP. Being a more specialized computation unit, it performs aspects of the signal processing algorithm with fewer transistors (less cost and power) compared to the more general purpose DSP. The blocks of hardware may be used to “accelerate” the performance of important specific function(s). Some architectures for hardware accelerators may be “programmable” via microcode or VLIW assembly. In the course of use, their functions may be accessed by calls to function libraries.

The hardware accelerator (HWA) module comprises an HWA input block to receive an input signal that is to be processed and instructions for processing the input signal; and, an HWA processing block to process the input signal according to the received instructions and to generate a resulting output signal. The resulting output signal may be transmitted as needed by an HWA output block.

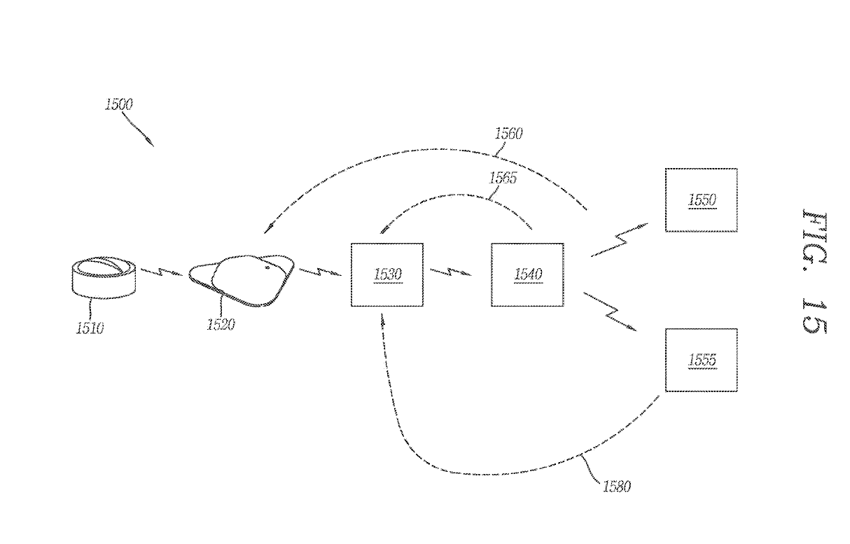

FIG. 14 provides a view of a block diagram of hardware in a receiver according to an aspect of the invention related to the high frequency signal chain. In FIG. 14, receiver 1400 includes receiver probes (for example in the form of electrodes 1411, 1412 and 1413) electrically coupled to multiplexer 1420. Also shown are high pass filter 1430 and low pass filter 1440 to provide for a band pass filter which eliminates any out-of-band frequencies. In the aspect shown, a band pass of 10 KHz to 34 KHz is provided to pass carrier signals falling within the frequency band. Example carrier frequencies may include, but are not limited to, 12.5 KHz and 20 KHz. One or more carriers may be present. In addition, receiver 1400 includes analog to digital converter 1450—for example, sampling at 500 KHz. The digital signal can thereafter be processed by the DSP. Shown in this aspect is DMA to DSP unit 1460 which sends the digital signal to dedicated memory for the DSP. The direct memory access provides the benefit of allowing the rest of the DSP to remain in a low power mode.

An example of a system of the invention is shown in FIG. 15. In FIG. 15, system 1500 includes a pharmaceutical composition 1510 that comprises an IEM. Also present in system 1500 is signal receiver 1520, such as the signal receiver illustrated in FIGS. 10 to 12. Signal receiver 1520 is configured to detect a signal emitted from the identifier of the IEM 1510. Signal receiver 1520 also includes physiologic sensing capability, such as ECG and movement sensing capability. Signal receiver 1520 is configured to transmit data to a patient's an external device or PDA 1530 (such as a smart phone or other wireless communication enabled device), which in turn transmits the data to a server 1540. Server 1540 may be configured as desired, e.g., to provide for patient directed permissions. For example, server 1540 may be configured to allow a family caregiver 1550 to participate in the patient's therapeutic regimen, e.g., via an interface (such as a web interface) that allows the family caregiver 1550 to monitor alerts and trends generated by the server 1540, and provide support back to the patient, as indicated by arrow 1560. The server 1540 may also be configured to provide responses directly to the patient, e.g., in the form of patient alerts, patient incentives, etc., as indicated by arrow 1565 which are relayed to the patient via PDA 1530. Server 1540 may also interact with a health care professional (e.g., RN, physician) 1555, which can use data processing algorithms to obtain measures of patient health and compliance, e.g., wellness index summaries, alerts, cross-patient benchmarks, etc., and provide informed clinical communication and support back to the patient, as indicated by arrow 1580.

Systems of the invention may include an external device which is distinct from the receiver (which may be implanted or topically applied in certain aspects), where this external device provides a number of functionalities. Such an apparatus can include the capacity to provide feedback and appropriate clinical regulation to the patient. Such a device can take any of a number of forms. By example, the device can be configured to sit on the bed next to the patient, e.g., a bedside monitor. Other formats include, but are not limited to, PDAs, phones, such as smart phones, computers, etc. The device can read out the information described in more detail in other sections of the subject patent application, both from pharmaceutical ingestion reporting and from physiological sensing devices, such as is produced internally by a pacemaker device or a dedicated implant for detection of the pill. The purpose of the external apparatus is to get the data out of the patient and into an external device. One feature of the external apparatus is its ability to provide pharmacologic and physiologic information in a form that can be transmitted through a transmission medium, such as a telephone line, to a remote location such as a clinician or to a central monitoring agency.

### Methods of Use

Aspects of the invention further include methods of using the compositions, such as those described above. Aspects of such methods include administering an ingestible composition to a subject, e.g., by self-administration or via the assistance of another, such as a health care practitioner. Such methods may include placing the ingestible composition in the mouth of a subject such that the subject swallows the ingestible composition. In this manner, the subject ingests the ingestible composition. Ingestible compositions may be employed with a variety of subjects. Subjects of interest include “mammals” including animals classified in the orders carnivore (e.g., dogs and cats), rodentia (e.g., mice, guinea pigs, and rats), and primates (e.g., humans, chimpanzees, and monkeys). In certain aspects, the subjects will be humans.

Following ingestion, the methods may include receiving a signal emitted from an ingestible composition, such as an IEM comprising ingestible composition, e.g., at a receiver, such as described above. In some instances, the received signal is a conductively transmitted signal.

Ingestible compositions may be employed in a variety of different applications. Applications of interest in which the ingestible composition comprises an IEM include, but are not limited to: monitoring patient compliance with prescribed therapeutic regimens; tailoring therapeutic regimens based on patient compliance; monitoring patient compliance in clinical trials; monitoring usage of controlled substances; monitoring the occurrence of a personal event of interest, such as the onset of symptoms, etc., and the like. Applications of interest are further described in PCT Application Serial No. PCT/US2006/016370 published as WO/2006/116718, PCT Application Serial No. PCT/US2007/082563 published as WO/2008/052136, PCT Application Serial No. PCT/US2007/024225published as WO/2008/063626, PCT Application Serial No. PCT/US2007/022257 published as WO/2008/066617, PCT Application Serial No. PCT/US2008/052845 published as WO/2008/095183, PCT Application Serial No. PCT/US2008/053999 published as WO/2008/101107, PCT Application Serial No. PCT/US2008/056296 published as WO/2008/112577, PCT Application Serial No. PCT/US2008/056299 published as WO/2008/112578, and PCT Application Serial No. PCT/US2008/077753 published as WO2009/042812; the disclosures of which applications is herein incorporated by reference.

### Kits

Also provided are kits that include one or more ingestible compositions, such as described above. In those aspects having a plurality of ingestible compositions, the ingestible compositions may be packaged in a single container, e.g., a single tube, bottle, vial, and the like, or one or more dosage amounts may be individually packaged such that certain kits may have more than one container of ingestible compositions. In certain aspects the kits may also include a receiver, such as reviewed above. In certain aspects, the kits may also include an external monitor device, e.g., as described above, which may provide for communication with a remote location, e.g., a doctors office, a central facility etc., which obtains and processes data obtained about the usage of the composition.

The subject kits may also include instructions for how to practice the subject methods using the components of the kit. The instructions may be recorded on a suitable recording medium or substrate. For example, the instructions may be printed on a substrate, such as paper or plastic, etc. As such, the instructions may be present in the kits as a package insert, in the labeling of the container of the kit or components thereof (i.e., associated with the packaging or sub-packaging) etc. In other aspects, the instructions are present as an electronic storage data file present on a suitable computer readable storage medium, e.g. CD-ROM, diskette, etc. In yet other aspects, the actual instructions are not present in the kit, but means for obtaining the instructions from a remote source, e.g. via the internet, are provided. An example of this aspect is a kit that includes a web address where the instructions can be viewed and/or from which the instructions can be downloaded. As with the instructions, this means for obtaining the instructions is recorded on a suitable substrate.

Some or all components of the subject kits may be packaged in suitable packaging to maintain sterility. In many aspects of the subject kits, the components of the kit are packaged in a kit containment element to make a single, easily handled unit, where the kit containment element, e.g., box or analogous structure, may or may not be an airtight container, e.g., to further preserve the sterility of some or all of the components of the kit.

It is to be understood that this invention is not limited to particular aspects described, as such may vary. It is also to be understood that the terminology used herein is for the purpose of describing particular aspects only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention.

The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

As will be apparent to those of skill in the art upon reading this disclosure, each of the individual aspects described and illustrated herein has discrete components and features which may be readily separated from or combined with the features of any of the other several aspects without departing from the scope or spirit of the present invention. Any recited method can be carried out in the order of events recited or in any other order which is logically possible.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and aspects of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary aspects shown and described herein. Rather, the scope and spirit of present invention is embodied by the appended claims.

What is claimed is:

1. An ingestible composition comprising:
an ingestible component;
an edible adhesive associated with the ingestible component, wherein the edible adhesive comprises:
a cellulose ether,
a tackifier and
a plasticizer; and
a highly-swellable polymeric film contacting the edible adhesive, wherein the highly-swellable polymeric film rapidly swells without disintegrating upon contact with an aqueous medium.

2. The ingestible composition according to claim 1, wherein the cellulose ether is an hydroxypropyl cellulose.

3. The ingestible composition according to claim 1, wherein the tackifier is a povidone.

4. The ingestible composition according to claim 1, wherein the plasticizer is glycerin.

5. The ingestible composition according to claim 1, wherein the edible adhesive consists of hydroxypropyl cellulose, povidone and glycerin.

6. The ingestible composition according to claim 1, wherein the highly-swellable polymeric film rapidly swells to ten times or greater in volume upon contact with an aqueous medium.

7. The ingestible composition according to claim 1, wherein the highly-swellable polymeric film comprises an ionic polymer.

8. The ingestible composition according to claim 7, wherein the ionic polymer is a polysaccharide.

9. The ingestible composition according to claim 8, wherein the polysaccharide is an alginate.

10. The ingestible composition according to claim 9, wherein the highly-swellable polymeric film further comprises a carboxyl functional group.

11. The ingestible composition according to claim 10, wherein the film comprises a polyacrylic acid.

12-14. (canceled)

15. The ingestible composition according to claim 1, wherein the ingestible component is an ingestible event indicator that comprises:
a support;
circuitry associated with the support;
a first material electrically coupled to the circuitry and associated with the support; and
a second material electrically coupled to the circuitry, wherein the second material is associated with the support and is electrically isolated from the first material;
wherein the first and second materials are selected to provide a voltage potential difference when in contact with a conducting fluid.

16. The ingestible composition according to claim 15, wherein the highly-swellable polymeric film covers at least a portion of one of the first material or second material.

17. The ingestible composition according to claim 16, wherein the highly-swellable polymeric film covers at least a portion of both the first material and the second material.

18-19. (canceled)

20. A system comprising:
an ingestible composition comprising:
an ingestible component;
an edible adhesive associated with the ingestible component, wherein the edible adhesive comprises a cellulose ether, a tackifier and a plasticizer; and
a highly-swellable polymeric film contacting the edible adhesive, wherein the highly-swellable polymeric film rapidly swells without disintegrating upon contact with an aqueous medium; and
a receiver configured to receive a communication associated with the ingestible component.

21. A method comprising combining:
an ingestible component,
an edible adhesive comprising:
a cellulose ether;
a tackifier; and
a plasticizer; and
a highly-swellable polymeric film, wherein the film rapidly swells without disintegrating upon contact with an aqueous medium.

22. The method according to claim 21, wherein the ingestible component is an ingestible event indicator.

23. The method according to claim 21, wherein the method comprises stably associating the ingestible component and the film with the adhesive.

24. The method according to claim 23, wherein the method comprises one or more protocols selected from the group consisting of laminating, pressing, stamping, extruding, molding, gluing and coating.

25. The method according to claim 24, wherein at least a portion of the method is automated.

26-30. (canceled)
